(19) United States
(12) Patent Application Publication
(10) Pub. No.: US 2014/0235717 A1
(43) Pub. Date: Aug. 21, 2014

(54) FOOD PRODUCTS, PREPARATION, AND THERAPEUTIC METHODS

(71) Applicant: John V. Schloss, Huntington, WV (US)

(72) Inventor: John V. Schloss, Huntington, WV (US)

(21) Appl. No.: 14/184,046

(22) Filed: Feb. 19, 2014

Related U.S. Application Data
(63) Continuation of application No. 13/580,461, filed on Aug. 22, 2012.
(60) Provisional application No. 61/306,838, filed on Feb. 22, 2010.

(51) Int. Cl. A61K 31/197; A23L 1/305; A23C 9/152

(52) U.S. Cl. CPC A61K 31/197; A23C 9/1526; A23L 1/3051
USPC 514/562

(57) ABSTRACT

A method is provided for the supplementation of milk, dairy products, meat products, and other food substances generally considered to be a major part of the Western diet, with substances capable of balancing the ratio of methionine and cysteine. Drug therapy with various carbamoyl thioesters or glutamate receptor antagonists can also be used, alone or in combination with dietary supplements and vitamins, to prevent or treat the pathology resulting from a Western diet.

This application claims priority to U.S. Provisional Application No. 61/306,838, filed 22 Feb. 2010, which is hereby incorporated by reference herein in its entirety.

The present invention provides methods for reducing the health risk associated with consumption of dairy products, meat, and other animal products by increasing their cysteine content and thereby decreasing the relative amounts of methionine and cysteine. Methods are also provided for treating or preventing the pathology associated with consumption of a diet high in dairy and/or animal products. Such a diet is commonly known as the Western diet.

The health risks associated with a Western diet have become clear: increased incidence of cancer, heart disease, diabetes and osteoporosis [Campbell and Campbell, The China Study, Benbella Books, Dallas, Tex., 2006]. Despite overwhelming evidence, the vast majority of individuals are not able to make dietary choices that significantly mitigate their risk. As such, there is a need to identify dietary supplements and therapeutic drug substances that can offset the consequences of poor dietary choices.

T. Colin Campbell described the cancer-promoting properties of the milk protein casein and the linkage between dietary animal proteins and various pathologic conditions [Campbell and Campbell, The China Study, Benbella Books, Dallas, Tex., 2006]. Although Campbell demonstrated that casein promotes cancer in laboratory animals, while soy protein and wheat gluten do not, he did not provide an explanation for these results. Further, he advocated a strict vegan diet to avoid the various pathologies associated with what is commonly known as the Western diet. Compliance with a strict vegan diet, which is a diet free of all food products derived from animal sources, is not possible for many individuals at risk or suffering from the pathologies associated with the Western diet.

Comparison of the amino acid compositions of casein and soy protein suggested an unexpected cause for the cancer-promoting properties of casein. Inspection of data reported by Rutherford and Moughan revealed that soy protein has substantially lower amounts of methionine and higher amounts of cysteine/cystine than casein [Rutherford and Moughan, Journal of Dairy Science, 81, 1998, 909-917]. In Example 1 it is shown that the ratio of methionine to cysteine is 7.7 in casein, compared to 1.2 in soy protein. The much higher methionine content of casein is responsible for an elevated methionine/cysteine ratio in milk of 4.5, while the soluble protein components of whey have a methionine/cysteine ratio closer to that of soy protein. FIG. 1 provides a graphic comparison of the methionine and cysteine content for casein, milk, whey, and soy protein.

The much higher content of methionine in cow's milk, relative to cysteine, is not seen in human milk. Data reported by Williamson revealed that human milk has a methionine/cysteine ratio of 0.71, compared to a ratio of 3.7 for cow's milk [Williamson, J. Biol. Chem., 156, 1944, 47-52]. Example 2 demonstrates that the relative amounts of methionine and cysteine in human milk proteins are more like soy protein than the proteins in cow's milk. FIG. 2 provides a graphic comparison of the relative amounts of these two amino acids in milk obtained from cow and human.

Example 3 extends the comparison of methionine and cysteine content to various meats, eggs, grains and legumes. Data reported by the Food and Agriculture Organization of the United Nations demonstrates that dairy and animal proteins are consistently higher in methionine and lower in cysteine than proteins derived from grains or legumes [FAO, A report of FAO/UN Joint Committee: Rome, Italy, P-84, 1981, ISBN 92-5-001102-4]. FIG. 3 provides a graphic comparison of the relative amounts of methionine and cysteine in various food products.

FIG. 4 illustrates the transsulfuration pathway and the metabolic relationship between methionine, homocysteine, cysteine, and glutathione. Methionine is an essential amino acid in that it can only be obtained from the diet. Cysteine can be synthesized from methionine or obtained in the diet. Conversion of methionine to cysteine involves homocysteine as an intermediate. Homocysteine and its oxidation products, homocysteine sulfinic acid and homocysteine sulfonic acid, are known to be toxic due to interaction with glutamate receptors [Shi et al., J Pharmacol Exp Ther, 305, 2003, 131-141]. Lower levels of cysteine and glutathione in autistic children have been linked to MTHFR gene polymorphism, with concomitant disturbances in the transsulfuration pathway and elevated homocysteine [Pasca et al., J Cell Mol Med, 13, 2008, 4229-4238]. The pathology associated with elevated homocysteine includes the same diseases associated with the Western diet, namely cancer, atherosclerosis, heart disease, diabetes, and osteoporosis [Carmel and Jacobsen, eds, Homocysteine in Health and Disease, Cambridge University Press, Cambridge, UK, 2001]. Glutamate receptors, including NMDA receptors, have now been found in various tissues in the periphery, such as pancreatic islet cells, kidney, heart, bone, lymphocyte, and skin cells [Miglio et al., Biochem Biophys Res Commun, 338, 2005, 1875-1883]. Glutamate receptor antagonists that prevent neurological damage in the central nervous system should also be effective in preventing and treating the toxicity caused by homocysteine and homocysteine oxidation products in the periphery [Faiman et al., U.S. Pat. No. 6,156,794, 2000; Schloss, U.S. Pat. No. 7,250,401, 2007]. Dietary cysteine would offset the pathology associated with dietary methionine in two ways. First, it would reduce the demand for conversion of methionine to cysteine and lower the intermediate levels of homocysteine. Second, conversion of cysteine to glutathione would have a compensatory effect on the toxic effect of homocysteine on glutamate receptors. Glutathione and S-nitrosoglutathione would compete with homocysteine and homocysteine oxidation products for glutamate receptors [Hermann et al., Neurochem Res, 25, 2000, 1119-1124].

Caloric restriction has long been known to increase lifespan in experimental animals. Recent studies in fruit flies have shown that dietary methionine restriction is essential to both an increase in lifespan and a reduction in fecundity [Grandison et al., Nature, 462, 2009, 1061-1064]. However, there is evidence that relative levels of dietary methionine and cysteine are important, rather than just the absolute levels of dietary methionine per se, in preventing the various disorders associated with the Western diet. N-acetyl-L-cysteine (NAC) has been proposed as an anti-obesity drug that improves insulin levels and insulin sensitivity in hyperinsulinemic patients with polycystic ovary syndrome [Kim et al., Exp Mol Med, 38, 2006, 162-172; Fulghesu et al., Fertil Steril, 77, 2002, 1128-1135]. Dietary NAC has also been identified as a potential cancer chemopreventative agent [Millea, Am Fam Physician, 80, 2009, 265-269]. NAC has also been shown to reduce atherosclerosis in apolipoprotein E-deficient mice [Shimada et al., Circ J, 73, 2009, 1337-1341]. NAC therapy of HIV+ patients causes a marked increase in immunological functions and plasma albumin concentrations [Breitkreutz et al., J Mol Med, 78, 2000, 55-62]. There was a positive correlation between plasma cysteine levels and bone mineral density in a study of 328 postmenopausal British women [Baines et al., Calcif Tissue Int, 81, 2007, 450-454].

The invention is based on the discovery that casein, a protein known to promote cancer in animal studies, has a dramatically elevated methionine to cysteine ratio relative to soy protein that does not promote cancer in the same animal model (EXAMPLE 1). Careful comparison of the amino acid compositions of casein and soy protein revealed that no other differences in their relative compositions could be used to explain the difference in their ability to promote cancer in experimental animal models. Casein has a methionine/cysteine ratio of 7.7, compared to a methionine/cysteine ratio of 1.2 in soy protein. Since cow's milk has a methionine/cysteine ratio of about 4, and whey has a methionine/cysteine ratio of about 1, the elevated ratio in cow's milk is due to the fact that casein is a major constituent.

Further, comparison of the amino acid compositions for proteins obtained from cow's milk with the amino acid compositions for proteins obtained from human milk revealed a similar difference (EXAMPLE 2). Whereas cow's milk has a methionine/cysteine ratio of about 4, human milk has a methionine/cysteine ratio of about 0.7. This indicates that the cancer-promoting activity seen with casein from cow's milk would not be expected for casein derived from human milk, since casein is the dominant protein in both. It also indicates that the elevated methionine/cysteine ratio is specific to neonatal nutrition in ruminant animals and is likely to be of no nutritional benefit to humans.

Comparison of the methionine/cysteine ratio in a number of animal-derived food products revealed a range of values from a low of 1.4 to a high of 7.0 (EXAMPLE 3). On average most foods derived from animal products have about twice the methionine content than cysteine. The methionine/cysteine ratio in a number of plant-derived food products revealed a range of values from a low of 0.6 to a high of 1.2. On average most foods derived from plant products have about equal amounts of methionine and cysteine. The relative levels of methionine and cysteine in plant-derived foods more closely match the levels found in human milk (EXAMPLE 2). The animal-derived foods with a higher relative methionine content have been identified in the China Study and other epidemiological studies as being linked to a higher incidence of various diseases associated with a Western diet, such as cancer, atherosclerosis, heart disease, diabetes, and osteoporosis.

The transsulfuration pathway and related metabolic transformations are illustrated in FIG. 4. Dietary methionine is converted to homocysteine, which is then converted to cystathionine by the enzyme cystathionine β-synthase. Deficiency in the enzyme cystathionine β-synthase is the primary cause of homocystinuria. Cystathionine is converted to cysteine by the enzyme cystathionine γ-lyase. Dietary cysteine cannot be converted to methionine. Cysteine can be converted to glutathione and taurine. Normally, the end products of a metabolic pathway will suppress their own formation by a process known as feedback inhibition. It is not clear whether cysteine, taurine, glutathione, or other cysteine-derived metabolic end products can suppress the formation of homocysteine or the conversion of methionine to cysteine. It is also possible that cysteine or cysteine-derived metabolic products could stimulate the conversion of homocysteine back to methionine through the action of tetrahydrofolate methyltransferase or betaine-homocysteine methyl transferase (BHMT). However, glutathione and S-nitrosoglutathione would compete with homocysteine and homocysteine oxidation products for an effect on glutamate receptors. The effect of homocysteine and homocysteine sulfinic acid on glutamate receptors, including metabotropic glutamate receptors and glutamate receptors of the NMDA subtype, are responsible for some of the pathology associated with this intermediate of the transsulfuration pathway [Hermann et al., Neurochem Res, 25, 2000, 1119-1124; Lipton et al., Proc Natl Acad Sci USA, 94, 1997, 5923-5928]. Faiman et al. and Schloss reported carbamoyl thioesters, in particular analogs of S-nitrosoglutathione, that would prevent pathology associated with glutamate receptors in the central nervous system [Faiman et al., U.S. Pat. No. 6,156,794, 2000; Schloss, U.S. Pat. No. 7,250,401, 2007]. However, use of these carbamoyl thioesters to treat diseases associated with the Western diet was not considered, since: 1) these diseases were not known to be linked to glutamate receptors; 2) glutamate receptors were not known to be in the periphery; and 3) the role of peripheral glutamate receptors in the pathology, and a link to diet-derived homocysteine, had not been established.

In the present invention, dairy products and other animal products can be made safer for human consumption by addition of N-acetyl-L-cysteine or other cysteine derivative or precursor. The amount added will reduce the methionine/cysteine ratio to a value closer to that found in human milk or plant-derived products. This addition can be made alone or in combination with other dietary substances known to lower homocysteine levels, such as folate, choline, betaine, vitamin B6, and vitamin B12 [Cuskelly et al., Am J Physiol Endocrinol Metab, 281, 2001, E1182-E1190]. Dietary supplements can also be added to the diet in a manner consistent with the foods being consumed, such that the combined methionine/cysteine ratio is lowered to a value closer to that found in human milk or plant derived products. These dietary supplements can be taken alone or in combination with other dietary substances known to lower homocysteine levels, such as folate, choline, betaine, vitamin B6, and vitamin B12. These dietary supplements can also be taken in combination with drugs that will treat the toxic effects of homocysteine, such as NMDA receptor antagonists or carbamoyl thioesters, as described by Faiman et al. and Schloss [Faiman et al., U.S. Pat. No. 6,156,794, 2000; Schloss, U.S. Pat. No. 7,250,401, 2007]. The toxic effects of excess dietary methionine can also be prevented or treated by use of NMDA receptor antagonists or carbamoyl thioesters, as described by Faiman et al. and Schloss, either alone, in various combinations, or in combination with the dietary supplements named herein.

### BRIEF DESCRIPTION OF THE FIGURES

FIG. 1. Comparison of the methionine (Met) and cysteine (Cys) contents of casein, milk, whey, and soy.

FIG. 2. Comparison of the methionine (Met) and cysteine (Cys) contents of cow's milk and human milk.

FIG. 3. Comparison of methionine (Met) and cysteine (Cys) contents of various foods.

FIG. 4. Interconversion of methionine, homocysteine, cysteine, and glutathione.

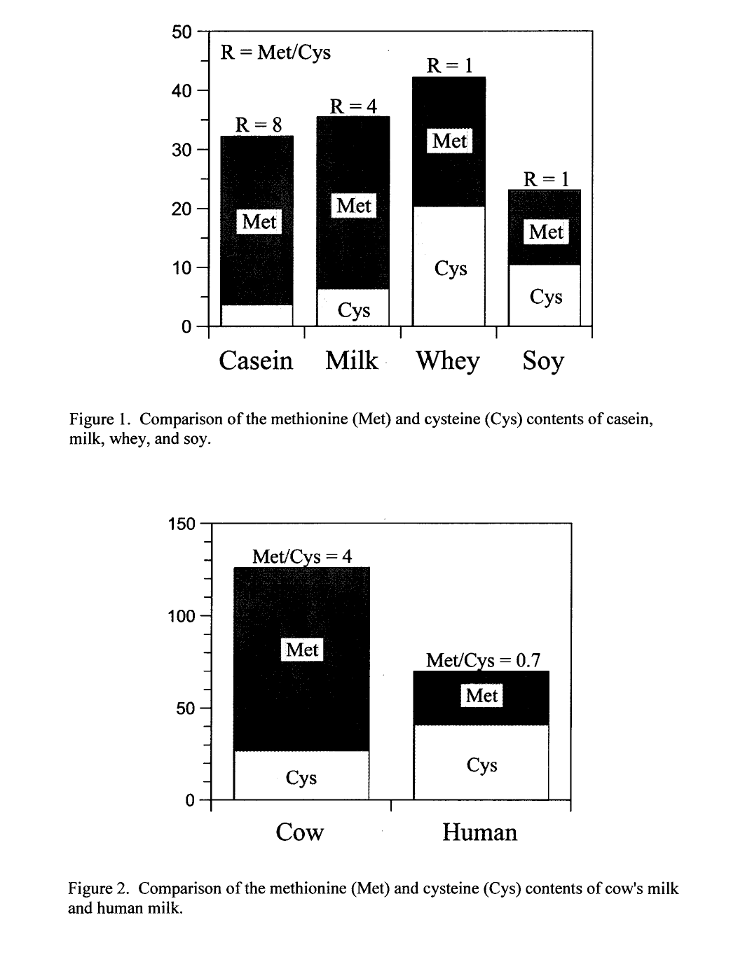

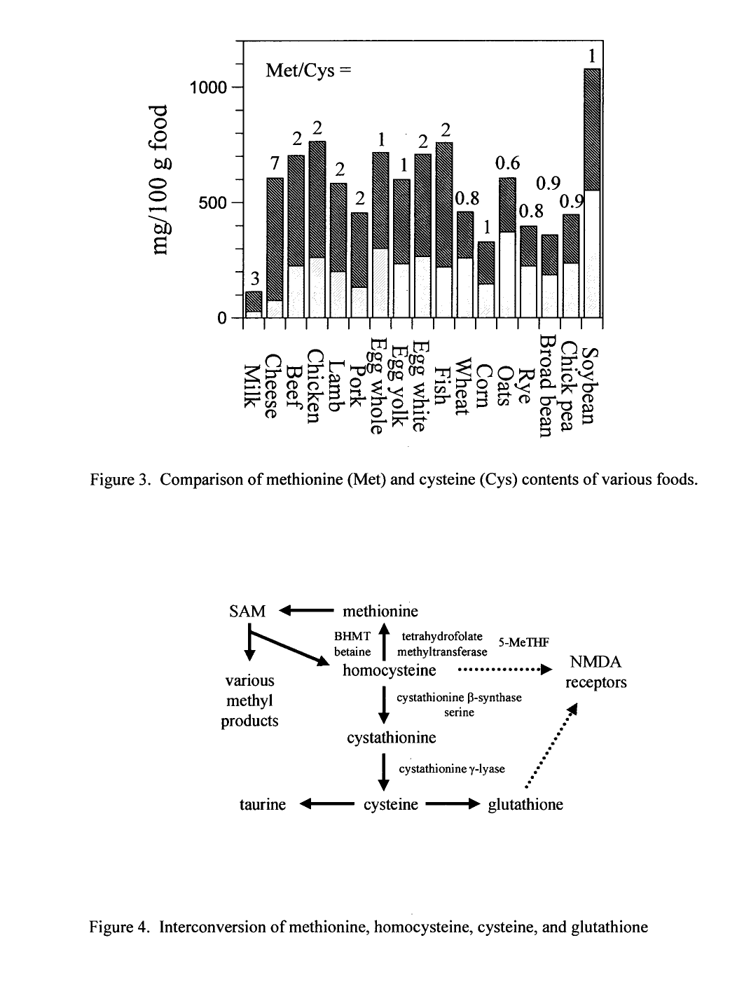

### DETAILED DESCRIPTION OF THE INVENTION

Supplementation of dairy products or other food products with cysteine, a cysteine derivative, or a cysteine precursor, such that the effective ratio of methionine/cysteine is within the range of 0.6 to 1.2, would match the range found in most plant-derived food products. The preferred amount to be supplemented would adjust the ratio to match the methionine/cysteine ratio seen in human milk, which is equal to 0.7 [Williamson, J. Biol. Chem., 156, 1944, 47-52]. The amount of compound to be added can be calculated from the following equations:

CPD Added=(CPD MW/Cys MW)[Met(R_{S}−R_{F})/R_{S}R_{F}]

CPD Added=(1/F_{Cys})[Met(R_{S}−R_{F})/R_{S}R_{F}]

where: CPD Added is the amount of compound to be supplemented in milligrams per 100 grams of food; CPD MW is the molecular weight of the compound to be supplemented per cysteine equivalent; Cys MW is the molecular weight of cysteine (121 g/mole); Met is the methionine content of the food expressed in milligrams per 100 grams of food; R_{S} is the starting ratio of methionine/cysteine; R_{F} is the final or desired ratio of methionine/cysteine; and F_{Cys} is the fractional weight of the substance to be supplemented that is comprised of cysteine.

The CPD MW for N-acetyl-L-cysteine (NAC) is 163 g/mol, so the ratio CPD MW/Cys MW is 1.35, which means 35% more NAC would be used to adjust the ratio of methionine/cysteine than cysteine. The equivalent molecular weights, CPD MW, for a number of different supplements are: 2-oxothiazolidine-4-carboxylic acid, 147 g/mole; cystine, 240/2=120 g/mole; glutathione, 307 g/mole; cysteinylglycine, 178 g/mole; and mercaptopyruvate, 120 g/mole. For small proteins with high cysteine content, like metallothionines, the fractional weight of the substance composed of cysteine, F_{Cys}, can be used. Metallothionines typically have molecular weights between 3,500 and 14,000 g/mole and consist of about 30% by weight cysteine. The F_{Cys} for metallothionines would be 0.30 and the effective CPD MW would be about 400 g/mole.

The methionine content of cow's milk, Met, has been reported to be 99 mg/100 cc (EXAMPLE 2) and 86 mg/100 grams (EXAMPLE 3). The starting methionine/cysteine ratio, R_{S}, in cow's milk has been reported to be 4.55 (EXAMPLE 1), 3.67 (EXAMPLE 2), and 3.07 (EXAMPLE 3). If NAC were used to supplement cow's milk to give a final methionine/cysteine ratio, R_{F}, of 0.7, then the amount of NAC to be added in mg/100 grams of milk can be calculated to be 140-160, 130-150, or 130-150 mg/100 grams of milk by use of the equation presented herein and the data from EXAMPLE 1, 2, or 3, respectively. To obtain cow's milk with a methionine/cysteine ratio equivalent to human milk, 1.3-1.6 grams of NAC should be added per liter of cow's milk.

To supplement an individual diet with sufficient cysteine, a cysteine derivative, or a cysteine precursor, such that the effective ratio of methionine/cysteine is within the range of 0.6 to 1.2, each of the animal-derived food products in the diet should be considered in calculating the total supplement required. The equation presented herein can be used to calculate the amount of supplement required, together with the amount of food consumed; the methionine/cysteine ratio for that food substance; and the dietary supplement to be used. Additional dietary substances known to lower homocysteine levels, such as folate, choline, betaine, vitamin B6, and vitamin B12, can be combined with the dietary supplement to further lower homocysteine levels [Cuskelly et al., Am J Physiol Endocrinol Metab, 281, 2001, E1182-E1190]. These dietary supplements can also be taken in combination with drugs that will treat the toxic effects of homocysteine, such as NMDA receptor antagonists or carbamoyl thioesters, as described by Faiman et al. and Schloss [Faiman et al., U.S. Pat. No. 6,156,794, 2000; Schloss, U.S. Pat. No. 7,250,401, 2007]. Other NMDA receptor antagonists can be taken alone or in combination with carbamoyl thioesters, other dietary supplements, and/or cysteine, a cysteine derivative, or a cysteine precursor. The NMDA receptor antagonists that can be used for preventing or treating toxicity resulting from a high ratio of methionine/cysteine include, but are not limited to, LY 274614, LY 235959, LY 233053, NPC 12626, carbamathione, the N-methyl or N-benzyl analogs of carbamathione, APS, CPP, CGS-19755, CGP-37849, CGP-39551,SDZ 220-581, S-nitrosoglutathione, amantadine, aptiganel, caroverine, dextrophan, dextromethorphan, fullerenes, gacyclidine (GK-11), ibogaine, ketamine, dizocilpine (MK-801), neramexane (MRZ 2/579), NPS 1506 (delucemine), phencyclidine, tiletamine, remacemide, acamprosate, arcaine, conantokin-G, eliprodil (SL 82-0715), haloperidol, ifenprodil, traxoprodil (CP-101,606), Ro 25-6981, aminocyclopropanecarboxylic acid (ACPC), 7-chlorokynurenic acid, D-cycloserine, gavestinel (GV-150526), GV-196771A, licostinel (ACEA 1021), MRZ-2/576, L-701,324, HA-966, ZD-9379, sodium nitroprusside, ebselen, disulfiram, argiotoxin636, Co 101244 (PD 174494, Ro 63-1908), despiramine, philanthotoxin343, Ro 04-5595, and NVP-AAM077.

The carbamoyl thioesters described by Faiman et al. and Schloss [Faiman et al., U.S. Pat. No. 6,156,794, 2000; Schloss, U.S. Pat. No. 7,250,401, 2007] can be used to prevent or treat the toxic effects of homocysteine resulting from a high dietary methionine/cysteine ratio or from other causes, such as, but not limited to, cystathionine β-synthase deficiency. Included among these compounds would be administration of an effective amount of a compound of Formula (I)

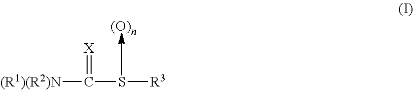

wherein:

a) R¹ and R² are individually H, (C₁-C₈)alkyl, aryl, heteroaryl, or R¹ and R² together with the nitrogen to which they are attached are a 4-8 membered ring optionally comprising 1, 2, or 3 additional heteroatoms selected from the group consisting of non-peroxide oxygen, sulfur, and N(Rₐ),

wherein each Rₐ is absent or is hydrogen, (C₁-C₈)alkyl, (C₁-C₈)alkanoyl, phenyl, benzyl, or phenethyl; and R³ is (C₁-C₈)alkyl, aryl, heteroaryl, or a glutathione derivative; or

b) R¹ and R³ together are a divalent ethylene or propylene chain and R² is (C₁C₈)alkyl, aryl, or heteroaryl; or

c) R¹ and R² together with the nitrogen to which they are attached are an azetidino, pyrrolidino, piperidino, hexamethyleneimin-1-yl, or heptamethylene-imin-1-yl ring, said ring being substituted on carbon by a substituent R_{b};

wherein R_{b} and R³ taken together are methylene (—CH₂—), ethylene (—CH₂CH₂—), or a direct bond; and wherein the ring comprising R_{b} and R³ is a five- or six-membered ring;

wherein any aryl or heteroaryl in R¹, R², or R³ may optionally be substituted with 1, 2, or 3 substituents selected from the group consisting of halo, nitro, cyano, hydroxy, (C₁-C₈)alkoxy, (C₁-C₈)alkanoyl, (C₂-C₈)alkanoyloxy, trifluoromethyl, trifluoromethoxy, and carboxy;

X is O or S; and

n is 0, 1, or 2;

or a pharmaceutically acceptable salt thereof.

Also included among these compounds would be administration of an effective amount of a compound of Formula (II):

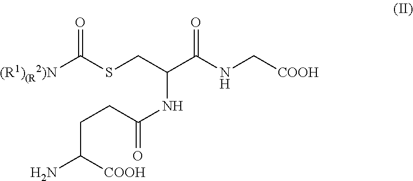

wherein:

a) R¹ and R² are individually H, (C₁-C₈)alkyl, aryl, or heteroaryl; or

b) R¹ and R² together with the nitrogen to which they are attached are a 4-8 membered ring optionally comprising 1, 2, or 3 additional heteroatoms selected from the group consisting of non-peroxide oxygen, sulfur, and N(Rₐ);

c) R¹ and R² together with the nitrogen to which they are attached are an azetidino, pyrrolidino, piperidino, hexamethyleneimin-1-yl, or heptamethylene-imin-1-yl ring;

wherein any aryl or heteroaryl in R¹ or R² may optionally be substituted with 1, 2, or 3 substituents selected from the group consisting of halo, nitro, cyano, hydroxy, (C₁-C₈)alkoxy, (C₁-C₈)alkanoyl, (C₂-C₈)alkanoyloxy, trifluoromethyl, trifluoromethoxy, carboxy; or

d) each Rₐ is absent or is hydrogen, (C₁-C₈)alkyl, (C₁-C₈)alkanoyl, phenyl, benzyl, or phenethyl;

or a pharmaceutically acceptable salt thereof.

The term “glutathione derivative” means a group of the formula:

H₂NCH(COOH)CH₂CH₂CONHCH(CH₂˜)CONHCH₂COOH.

Specific and preferred values listed below for radicals, substituents, and ranges, are for illustration only and they do not exclude other defined values or other values within defined ranges for the radicals and substituents.

Specifically, (C₁-C₈)alkyl can be methyl, ethyl, propyl, isopropyl, butyl, iso-butyl, sec-butyl, pentyl, 3-pentyl, hexyl, heptyl, octyl, cyclohexyl, (C₁-C₈)alkoxy can be methoxy, ethoxy, propoxy, isopropoxy, butoxy, iso-butoxy, sec-butoxy, pentoxy, isopropoxy, butoxy, iso-butoxy, sec-butoxy, pentoxy, 3-pentoxy, hexyloxy, heptyloxy, or octyloxy; (C₁-C₈)alkanoyl can be acetyl, propanoyl, butanoyl, isobutanoyl, pentanoyl, hexanoyl, heptanoyl, or octanoyl, and (C₂-C₈)alkanoyloxy can be acetoxy, propanoyloxy, butanoyloxy, isobutanoyloxy, pentanoyloxy, hexanoyloxy, heptanoyloxy, or octanoyloxy. Aryl can be phenyl, indenyl, or naphthyl. Heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl (or its N-oxide), thienyl, pyrimidinyl (or its N-oxide), indolyl, isoquinolyl (or its N-oxide) or quinolyl (or its N-oxide).

Preferably, alkyl is (C₁-C₄)alkyl and aryl is phenyl.

A specific group of compounds are compounds of Formula (I) wherein R¹ and R² together with the nitrogen to which they are attached are a ring selected from the group consisting of azetidino, pyrrolidino, piperidino, hexamethyleneimin-1-yl, and heptamethyleneimin-1-yl. Another specific group of compounds are compounds of Formula (I) wherein R¹ and R³ together are a divalent ethylene or propylene chain and R² is (C₁-C₈)alkyl, (C₆-C₁₂)aryl, or heteroaryl. A third specific group of compounds are compounds of Formula (I) wherein R¹ and R² together with the nitrogen to which they are attached are an azetidino, pyrrolidino, piperidino, hexamethyleneimin-1-yl or heptamethyleneimin-1-yl ring, said ring being substituted on carbon by a substituent R_{b}; wherein R_{b} and R³ taken together are a divalent ethylene or propylene chain.

A preferred group of compounds are compounds of Formula (I) wherein R¹ and R² are individually (C₁-C₈)alkyl, or (C₆-C₁₂)aryl, hydrogen, or a glutathione derivative; X is O or S; n is 0, 1 or 2 or a pharmaceutically acceptable salt thereof.

Another preferred group of compounds are compounds of Formula (I) wherein R³ is aryl or heteroaryl and n is 0.

Another preferred group of compounds are compounds of Formula (II) wherein R¹ and R² are individually (C₁-C₈)alkyl, or aryl; X is O; n is 0. When both R¹ and R² are ethyl (C₂H₅), this compound is referred to as carbamathione.

These compounds can be administered separately or in combination with cysteine, a cysteine derivative, a cysteine precursor, folate, choline, betaine, vitamin B6, vitamin B 12, or another NMDA receptor antagonist, including, but not restricted to, LY 274614, LY 235959, LY 233053, NPC 12626, carbamathione, the N-methyl or N-benzyl analogs of carbamathione, AP5, CPP, CGS-19755, CGP-37849, CGP-39551, SDZ 220-581, S-nitrosoglutathione, amantadine, aptiganel, caroverine, dextrophan, dextromethorphan, fullerenes, gacyclidine (GK-11), ibogaine, ketamine, dizocilpine (MK-801), neramexane (MRZ 2/579), NPS 1506 (delucemine), phencyclidine, tiletamine, remacemide, acamprosate, arcaine, conantokin-G, eliprodil (SL 82-0715), haloperidol, ifenprodil, traxoprodil (CP-101,606), Ro 25-6981, aminocyclopropanecarboxylic acid (ACPC), 7-chlorokynurenic acid, D-cycloserine, gavestinel (GV-150526), GV-196771A, licostinel (ACEA 1021), MRZ-2/576, L-701,324, HA-966, ZD-9379, sodium nitroprusside, ebselen, disulfiram, argiotoxin636, Co 101244 (PD 174494, Ro 63-1908), despiramine, philanthotoxin343, Ro 04-5595, and NVP-AAM077.

In one embodiment the invention provides a method for preparing a modified dairy or food product that has an effective ratio of methionine/cysteine of less than 1.5, comprising adding cysteine, a cysteine derivative, or a cysteine precursor to a starting dairy or food product to provide the modified dairy or food product that has an effective ratio of methionine/cysteine of less than about 1.5. In one embodiment the starting dairy or food product has an effective ratio of methionine/cysteine of at least about 9.0. In one embodiment the starting dairy or food product has an effective ratio of methionine/cysteine of at least about 5.0. In one embodiment the starting dairy or food product has an effective ratio of methionine/cysteine of at least about 3.0. In one embodiment the starting dairy or food product comprises cheese, milk, ice cream, or meat. In one embodiment the starting dairy or food product comprises casein. In one embodiment casein has been added to the starting dairy or food product.

### Example 1

Data reported by Rutherford and Moughan were analyzed for differences in the amino acid compositions of casein from cow's milk, cow's milk, the whey from cow's milk, and soy protein [Rutherford and Moughan, Journal of Dairy Science, 81, 1998, 909-917]. These data are presented in Table 1.

TABLE 1
Comparison of casein, milk, whey, and soy.

| | Met | Cys | |
|---|---|---|---|
| | (mg/g protein) | (mg/g protein) | Met/Cys |
| Casein | 28.5 | 3.7 | 7.70 |
| Milk | 29.1 | 6.4 | 4.55 |
| Whey | 21.8 | 20.4 | 1.07 |
| Soy | 12.6 | 10.5 | 1.20 |

The data of Rutherford and Moughan reveal that the only major difference in the amino acid compositions of casein and soy protein are in the levels of methionine and cysteine. Casein contains 2.3 times the methionine content of soy proteins on a weight basis and only 35% of the cysteine content of soy protein. Together these differences account for a 6.6-fold change in the ratio of methionine/cysteine from soy protein (Met/Cys=1.2) to casein (Met/Cys=7.7). The high ratio of methionine/cysteine in milk is due to its casein content, since the ratio in whey is similar to soy protein.

### Example 2

The data of Williamson were analyzed for differences in the amino acid composition of cow's milk and human milk [Williamson, J. Biol. Chem., 156, 1944, 47-52]. These data are presented in Table 2.

TABLE 2
Comparison of cow's milk and human milk.

| | Met | Cys | |
|---|---|---|---|
| | (mg/100 cc milk) | (mg/100 cc milk) | Met/Cys |
| Cow's milk | 99 | 27 | 3.67 |
| Human milk | 29 | 41 | 0.71 |

The data of Williamson reveal that the only major difference in the amino acid compositions of cow's milk and human milk are in the levels of methionine and cysteine. Cow's milk contains 3.4 times the methionine content of human milk on a weight basis and only 66% of the cysteine content of human milk. Together these differences account for a 5.1-fold change in the ratio of methionine/cysteine from human milk (Met/Cys=0.71) to cow's milk (Met/Cys=3.67).

### Example 3

Data reported by the Food and Agriculture Organization of the United Nations were analyzed for differences in the methionine and cysteine content of various foods [FAO, A report of FAO/UN Joint Committee: Rome, Italy, P-84, 1981, ISBN 92-5-001102-4]. These data are presented in Table 3.

The highest ratio of methionine/cysteine was seen for cheese made from cow's milk, which has an amino acid composition similar to casein. The value reported for cow's milk (Met/Cys=3.07) is somewhat lower than the value reported by Williamson (Met/Cys=3.67) or by Rutherford and Moughan (Met/Cys=4.55). However, despite this difference in the values reported by these three independent sources, the values for methionine are consistently much higher than cysteine. The methionine values were substantially higher than cysteine for all foods derived from animal sources. The methionine/cysteine ratio varied from a low of 1.38 for whole eggs, to 1.91-2.45 for various meat products (beef, chicken, lamb, or pork). By contrast, the values for various plant-derived foods were much lower. The methionine/cysteine ratio varied from a high of 1.24 for corn to a low of 0.59 for wheat and with most plant food products below a value of 1 (oats, rye, broad bean, chickpea, and soybean). The value reported for soybean (Met/Cys=0.95) was similar to, but slightly lower than, the value reported by Rutherford and Moughan (Met/Cys=1.2).

TABLE 3
Comparison of the methionine and cysteine content of various foods.

| | Met | Cys | |
|---|---|---|---|
| | (mg/100 g food) | (mg/100 g food) | Met/Cys |
| Milk | 86 | 28 | 3.07 |
| Cheese | 530 | 76 | 6.97 |
| Beef | 478 | 226 | 2.12 |
| Chicken | 502 | 262 | 1.92 |
| Lamb | 383 | 200 | 1.91 |
| Pork | 321 | 133 | 2.41 |
| Eggs whole | 416 | 301 | 1.38 |
| Egg yolk | 364 | 235 | 1.55 |
| Egg white | 441 | 267 | 1.65 |
| Fish | 539 | 220 | 2.45 |
| Wheat | 196 | 332 | 0.59 |
| Corn | 182 | 147 | 1.24 |
| Oats | 234 | 372 | 0.63 |
| Rye | 172 | 225 | 0.76 |
| Broad bean | 172 | 187 | 0.92 |
| Chickpea | 209 | 238 | 0.88 |
| Soybean | 525 | 552 | 0.95 |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A dairy or food product that has been modified by addition of cysteine, a cysteine derivative, or a cysteine precursor such that the effective ratio of methionine/cysteine is less than about 1.5.

2. The dairy or food product of claim 1 wherein the cysteine derivative is selected from the group consisting of L-cysteine, L-cystine, N-acetyl-L-cysteine, N-acetyl-L-cysteine disulfide, L-2-oxothiazolidine-4-carboxylate, 3-mercaptopyruvate, L-glutathione, cysteinylglycine, metallothionines, cysteine-containing peptides or proteins, and cystathionine, or a pharmaceutically acceptable salt thereof or pharmaceutically acceptable ester thereof, such as, but not limited to, an acetyl, palmitoyl, or ethyl ester.

3. The dairy or food product of claim 1 where the cysteine derivative is N-acetyl-L-cysteine, or a pharmaceutically acceptable salt thereof or pharmaceutically acceptable ester thereof, such as, but not limited to, an acetyl, palmitoyl, or ethyl ester, such as N, S-diacetyl-L-cysteine, N-acetyl-S-palmitoyl-L-cysteine, or N, S-diacetyl-L-cysteine ethyl ester.

4. The dairy or food product of claim 1 wherein methionine/cysteine ratio is about 0.7.

5. A method for preparing a modified dairy or food product that has an effective ratio of methionine/cysteine of less than 1.5, comprising adding cysteine, a cysteine derivative, or a cysteine precursor to a starting dairy or food product to provide the modified dairy or food product that has an effective ratio of methionine/cysteine of less than about 1.5.

6. The method of claim 5 wherein the cysteine derivative is selected from the group consisting of L-cysteine, L-cystine, N-acetyl-L-cysteine, N-acetyl-L-cysteine disulfide, L-2-oxothiazolidine-4-carboxylate, 3-mercaptopyruvate, L-glutathione, cysteinylglycine, metallothionines, cysteine-containing peptides or proteins, and cystathionine, or a pharmaceutically acceptable salt thereof or pharmaceutically acceptable ester thereof, such as, but not limited to, an acetyl, palmitoyl, or ethyl ester.

7. The method of claim 5 where the cysteine derivative is N-acetyl-L-cysteine or a pharmaceutically acceptable salt thereof or pharmaceutically acceptable ester thereof, such as, but not limited to, an acetyl, palmitoyl, or ethyl ester, such as N,S-diacetyl-L-cysteine, N-acetyl-S-palmitoyl-L-cysteine, or N, S-diacetyl-L-cysteine ethyl ester.

8. The method of claim 5 wherein the modified dairy or food product has an effective ratio of methionine/cysteine of less than about 0.7.

9. A method for preventing or treating a pathology associated with consumption of a Western diet in a human or a genetic disorder resulting in elevated levels of homocysteine in a human comprising supplementing the diet of the human with cysteine, a cysteine derivative, or a cysteine precursor, such that the effective ratio of total methionine/total cysteine is less than about 1.5.

10. The method of claim 9 wherein the cysteine derivative is selected from the group consisting of L-cysteine, L-cystine, N-acetyl-L-cysteine, N-acetyl-L-cysteine disulfide, L-2-oxothiazolidine-4-carboxylate, 3-mercaptopyruvate, L-glutathione, cysteinylglycine, metallothionines, cysteine-containing peptides or proteins, and cystathionine, or a pharmaceutically acceptable salt thereof or pharmaceutically acceptable ester thereof, such as, but not limited to, an acetyl, palmitoyl, or ethyl ester.

11. The method of claim 9 wherein the cysteine derivative is N-acetyl-L-cysteine or a pharmaceutically acceptable salt thereof or pharmaceutically acceptable ester thereof, such as, but not limited to, an acetyl, palmitoyl, or ethyl ester, such as N,S-diacetyl-L-cysteine, N-acetyl-S-palmitoyl-L-cysteine, or N, S-diacetyl-L-cysteine ethyl ester.

12. The method of any claim 9 wherein the methionine/cysteine ratio is about 0.7.

13-16. (canceled)

17. A method for treating a pathology associated with the Western diet in a human or a genetic disorder resulting in elevated homocysteine in a human comprising administering to the human 1) one or more cysteine derivatives as described in claim 2, and optionally 2) one or more nutraceuticals selected from the group consisting of folate, betaine, choline, vitamin B6, and vitamin B12.

18. The method of claim 9 wherein the pathology associated with consumption of a Western diet or the genetic disorder resulting in elevated homocysteine is selected from the group consisting of cancer, atherosclerosis, osteoporosis, autism, Asperger's, diabetes, and coronary heart disease.

19-22. (canceled)

23. A product prepared by the method of claim 5.

24. The product of claim 23 which comprises milk.

25. The product of claim 23 which comprises cheese.

26. The product of claim 23 which comprises ice cream.

27. The product of claim 23 which comprises meat.

28. The product of claim 23 which comprises casein.
